(19) United States
(12) Patent Application Publication
(10) Pub. No.: US 2004/0092932 A1
(43) Pub. Date: May 13, 2004

(54) Adjustable surgical templates

(76) Inventors: Carl-Eric Aubin, St-Lambert (CA); Florent Salako, Montreal (CA); Hubert Labelle, Montreal (CA); Clement Fortin, Ile Bizard (CA)

Correspondence Address: OGILVY RENAULT, 1981 MCGILL COLLEGE AVENUE, SUITE 1600, MONTREAL, QC, H3A2Y3, CA

(21) Appl. No.: 10/428,100

(22) Filed: May 2, 2003

Related U.S. Application Data
(63) Continuation of application No. PCT/CA00/01317, filed on Nov. 3, 2000.

(51) Int. Cl. A61B 17/56

(52) U.S. Cl. 606/61

(57) ABSTRACT

A surgical template (30) adjustable in conformity to specific geometric parameters of an intraoperatively reachable bone surface. The template (30) comprises a number of bone-engaging elements (40, 48 and 50) adapted to be preoperatively adjusted and maintained in a predetermined configuration to match corresponding predetermined contact points on the bone surface for allowing the surgical template (30) to be readily intraoperatively registered in a predetermined position on the bone surface. The template (30) also includes a guide (36) adapted to be preoperatively adjusted according to the geometric parameters of the bone for guiding a surgical tool in a predetermined direction to a specific location on the bone surface when the surgical template is registered thereon.

### RELATED APPLICATIONS

This is a continuation of International Patent Application No. PCT/CA00/01317, filed Nov. 3, 2000.

### BACKGROUND OF THE INVENTION

1. Field of the Invention

The present invention relates to surgical instrumentation and, more particularly, pertains to a surgical template which can be adjusted in accordance with the specific geometry of a selected bone structure.

2. Description of the Prior Art

It is known to drill holes in bone in order to receive fastening elements used to anchor instrumentation within a patient's body. For instance, pilot holes can be drilled into selected vertebrae to receive pedicle screws used for anchoring internal instrumentation systems to a patient's spinal column. The drilling direction must be in alignment with a pedicle axis of each selected vertebra and not be allowed to deviate off axis. Slight deviations of the drilling direction could injure the nerve roots or spinal cord.

Therefore, methods and systems to prevent nerve roots and spinal cords from being injured have been developed. For instance, Radermacher k., and Staudte H. W. disclose in “Computer Assisted Orthopedic Surgery by means of Individuals Templates”, Rau G. 1994, Medical Robotics and Computer Assisted Surgery, pp.42-48, a disposable or one-time use surgical template designed on the basis of preoperative CT image data of a patient's bone. The surgical template has a base-contact surface that is generated from the collected image data so as to be complementary to an intraoperatively reachable surface of the patient's bone. Therefore, the template can be intraoperatively fitted on the patient's bone in a predetermined planned position and orientation. Guides, such as jig bush, for the guidance of a surgical tool can be integrated to the template in a predetermined position and orientation with respect thereto. The surgeon is, thus, guided intraoperatively according to the preoperative plan by simply fitting the template in registry on the bone.

Although the individual template described in the above reference is effective, it has been found that the surface-to-surface fitting method thereof is sensible to registration errors which could result from the modeling of the bone, the manufacturing of the template, or the presence of tissues on the exposed surface of the bone. Furthermore, Radermacher's individual template is limited to a single utilization.

### SUMMARY OF THE INVENTION

It is therefore an aim of the present invention to provide a new surgical template which is adapted to be customized to the geometry of a patient's vertebra in order to improve the accuracy of the surgery and reduce the risks associated therewith.

It is also an aim of the present invention to provide such a surgical template which is adjustable for allowing the same to be reused on different patients.

It is a further aim of the present invention to provide a surgical template that will contribute to reduce the time of some surgical interventions.

It is a still further aim of the present invention to provide a method in which a surgical template is preoperatively adjusted in conformity to geometric parameters of a bone surface, thereby eliminating the need for imaging systems during the medical procedure.

Therefore, in accordance with the present invention, there is provided a surgical template adjustable in conformity to geometric parameters of an intraoperatively reachable bone surface. The surgical template comprises positioning means including a number of bone-engaging elements adapted to be preoperatively adjusted and fixed in a predetermined configuration to match corresponding predetermined contact points on the bone surface for allowing the surgical template to be readily intraoperatively registered in a predetermined position on the bone surface. A guide forming part of the surgical template is provided for guiding a surgical tool in a predetermined direction to a specific location on the bone surface when the surgical template is in said predetermined position.

In accordance with a further general aspect of the present invention, there is provided a method of orienting a surgical tool relative to a bone surface, wherein the surgical tool must contact a specific location on the bone surface at a predetermined angle, comprising the steps of: generating a three dimensional computer model of the bone surface, providing a surgical template having bone-engaging elements and a guide; given the specific geometrical parameters of the bone surface, adjusting said bone-engaging elements in a predetermined configuration in which said bone-engaging elements match predetermined contact points on said bone surface for allowing said surgical template to be registered in a unique preoperatively determined position on said bone surface; given said preoperatively determined position, adjusting the orientation of said guide according to the specific geometrical parameters of the bone surface and the task to be performed; localizing said templates on said bone surface until a perfect match is obtained with said bone-engaging elements abutting against said corresponding predetermined contact points on the bone surface, thereby automatically orienting said guide relative to said bone surface for guiding said surgical tool to contact the specific location on the bone surface at the predetermined angle.

### BRIEF DESCRIPTION OF THE DRAWINGS

Having thus generally described the nature of the invention, reference will now be made to the accompanying drawings, showing by way of illustration a preferred embodiment thereof, and in which:

FIG. 1 is a posterior elevational view of a vertebral body;

FIG. 2 is a transversal view of the vertebral body with a pedicle screw implanted therein;

FIG. 3 is a front perspective view of an adjustable surgical template in accordance with a first embodiment of the present invention;

FIG. 4 is a rear perspective view of the surgical template of FIG. 3; and

FIG. 5 is a side elevational view of the surgical template maintained in position on a selected vertebra by means of a surgical clamping tool.

### DESCRIPTION OF THE PREFERRED EMBODIMENTS

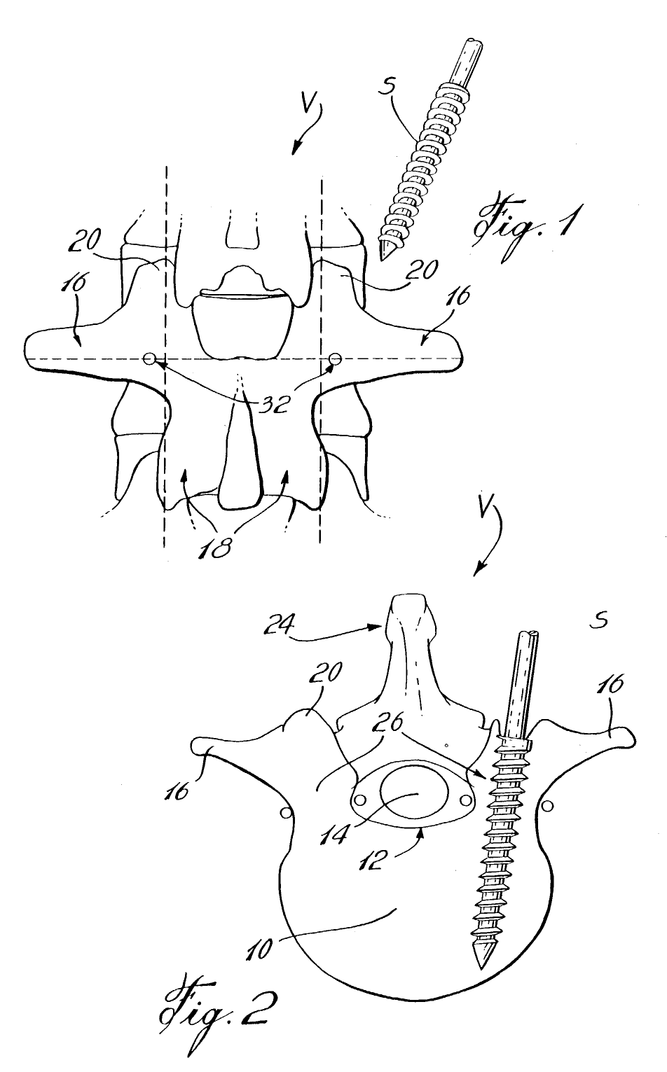

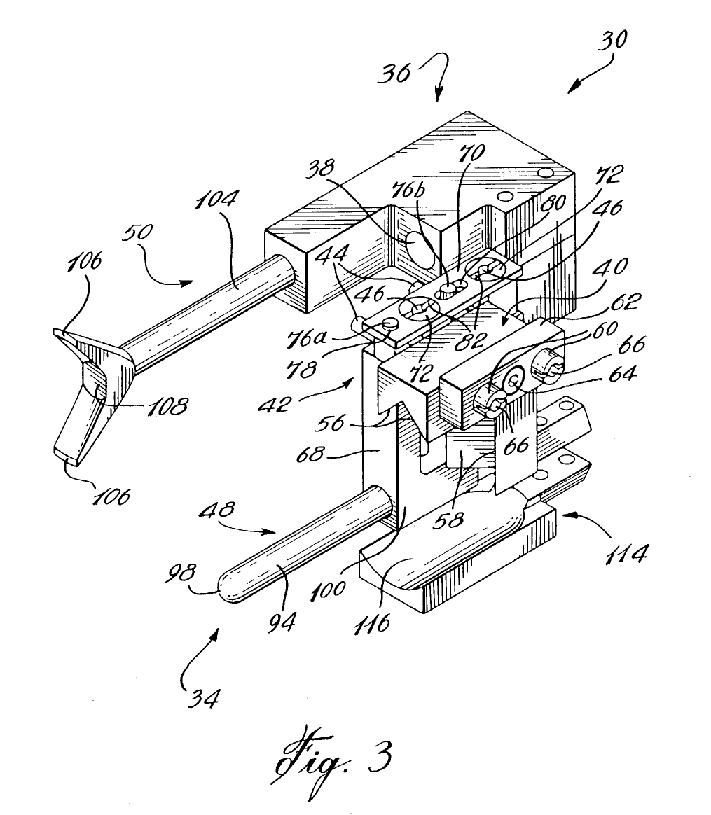

As illustrated in FIGS. 1 and 2, a vertebra V generally include a vertebral body 10 defining a spinal canal 12 with a spinal cord 14 therein, a pair of transverse processes 16 extending from opposed sides of the vertebral body 10, inferior and superior articular processes 18 and 20, a spinal process 24, and a pedicle 26 located at each side of the vertebral body 10. As seen in FIG. 2, the pedicle axis is the suitable direction for implanting a screw S in the vertebra V.

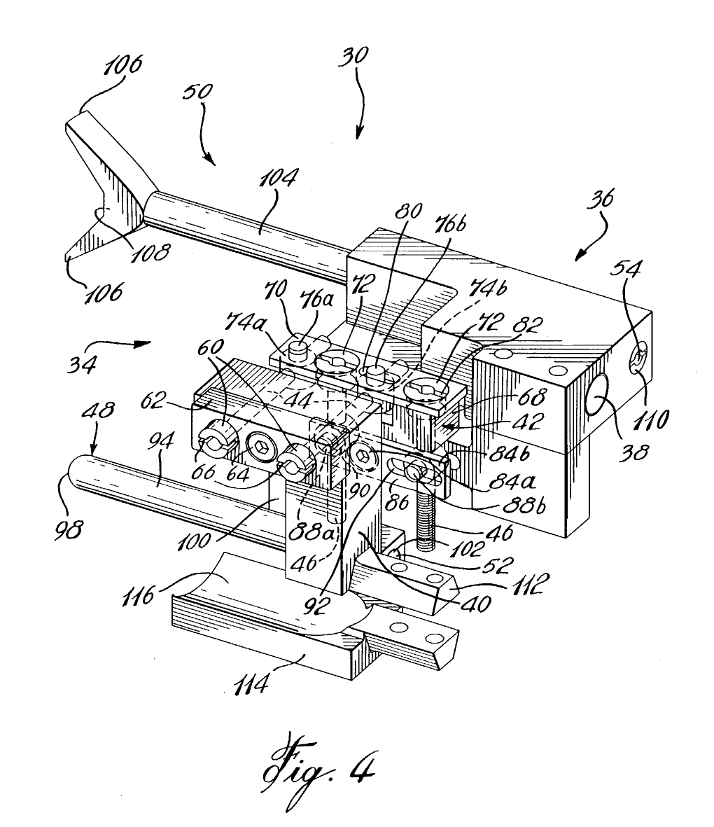

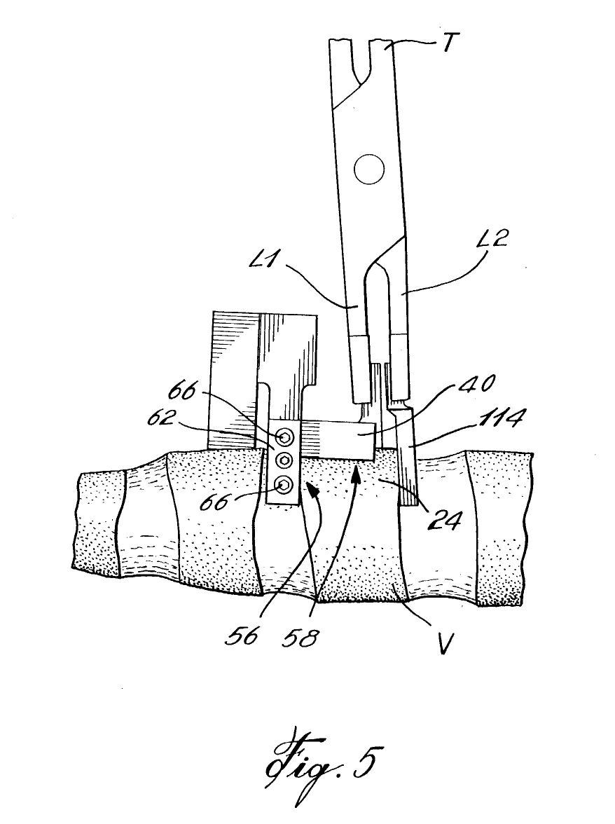

Now referring to FIGS. 3 and 4, a surgical template 30 adjustable to the specific geometry of a selected vertebra and embodying the elements of the present invention will be described.

The surgical template 30 is designed and adjusted on the basis of preoperative image data of the patient's vertebra V in which a screw S (see FIG. 2) is to be implanted. To do so, image data of the patient's vertebra are first gathered using radiant energy means, such as a conventional CT scanning device. According to a procedure of the present invention, an appropriate number of 1 mm CT image “slices” (two-dimensional image taken in a transverse plane) of the patient's vertebra V are collected. The number of slices that are taken can vary depending on the dimensions of the vertebra, but enough slices must be taken for allowing the generation of an accurate three-dimensional computer model of the vertebra.

The so collected image data are then provided to an image processing system for use in generating a three-dimensional computer model of the vertebra V. The system may comprise a computer and a CAD software for reading the image data stored on the memory of the computer and generating a three-dimensional anatomical model of the vertebra V from the image data.

The formed geometric computer model of the vertebra V is then used in the creation and the adjustment of the surgical template 30. More particularly, the surface reconstruction of the posterior surface (FIG. 1) of the vertebra V is used to compute the entry point 32 of the screw S in the vertebra V as well as the optimum drilling direction and the limit angles based on an inverse projection of the limits of the selected pedicle on the transverse and sagital planes of the vertebra V, as is known in the art. The optimum drilling direction can, for instance, be provided by the surgeon by clicking two points on the computer model of the vertebra, the two points defining a trajectory line (i.e. the drilling axis). The entry point 32 can then be computed by a an appropriate software. The surface reconstruction is also used to ascertain the spatial coordinates of a number of reference points on the posterior surface. Given the coordinates of these reference points, the surgical template 30 will be adjusted so as to allow the same to be readily intraoperatively located in a unique predetermined position on the vertebra. Hence, the planned drilling direction will be automatically intraoperatively reproduced by simply putting the surgical template 30 on the vertebra V, as will be seen hereinafter.

As seen in FIG. 3, the surgical template 30 generally comprises a positioning assembly 34 and a drill guide 36 defining a passage 38 for guiding a drill bite of a drill tool (not shown) during a surgical intervention.

The positioning assembly 34 includes a reference bone-engaging element 40 connected to the drill guide 36 via an intermediate support 42. A first pair of parallel coplanar setscrews 44 are mounted to the reference bone-engaging element 40 and extend in a same transversal plane with respect thereto for adjusting the position of the intermediate support 42 relative to the reference bone-engaging element 40 in the plane of the setscrews 44. A second pair of coplanar setscrews 46 are mounted to the intermediate support 42 laterally of the reference bone-engaging element 40 at right angles with respect to the first pair of setscrews 44 for adjusting the position of the guide 36 relative to the intermediate support 42 in the plane of the second pair of setscrews 46.

As shown in FIG. 3, the positioning assembly 34 further includes first and second adjustable bone-engaging elements 48 and 50 respectively mounted to the intermediate support 42 and the guide 36. First and second additional setscrews 52 and 54 (FIG. 4) are respectively provided for linearly displacing the first and second adjustable bone-engaging elements 48 and 50 relative to the intermediate support 42 and the guide 36, respectively.

The reference bone-engaging element 40 is generally L-shaped and includes a first pair of bone-engaging surfaces 56 adapted to be placed on a top surface of the spinal process 24 of the vertebra V and a second pair of bone-engaging surfaces 58 adapted to be placed on the posterior surface of the spinal process 24. The conception of the first and second pairs of bone-engaging surfaces 56 and 58 is based on the tangential points between the spinal process 24 and four predetermined vertical and horizontal planes. The two vertical planes, which corresponds to the second pair of bone-engaging surfaces 58 have an orientation of +45° and −45° relative to the sagital plane of the vertebra V. The horizontal planes, which corresponds to the first pair of bone-engaging elements 56, have an orientation of +45° and −45° relative to a horizontal plane of the vertebra V.

Based on the computer model of the vertebra V, four contact points on the operatively reachable surface of the spinal process 24 are calculated with tangential points between the spinal process 24 and the first and second pair of bone-engaging surfaces 56 and 58. The spatial coordinates of a first additional contact point on the inferior articular process 18 and of a second additional contact point on the posterior surface of one of the transversal processes 16 of the vertebra V are also determined.

Given the coordinates of these contact points, the software used to manipulate the computer model of the vertebra V calculates the length or the number of turns (based on the pitch thereof) that each setscrew 44, 46, 52 and 54 must be turned to fix the bone-engaging elements 40, 48 and 50 in a desired configuration wherein the bone-engaging elements 40, 48, 50 match the predetermined contact points on the vertebra V in a complementary fashion, thereby allowing the surgical template 30 to be precisely adjusted and subsequently introperatively registered in a unique predetermined position on the vertebra V. Therefore, by preoperatively establishing the drilling direction and the coordinates of the entry point 32, and given the calculated predetermined position of the surgical template 30 relative to the vertebra V, the orientation that the guide 36 must have relative to the reference bone-engaging element 40 to guide the drilling tool to the entry point 32 at the desired angle can be readily computed by the software, thereby allowing the guide to be preoperatively adjusted by operation of the setscrews 44 and 46 so that upon positioning of the surgical template 30 on the vertebra V in its predetermined position, the preoperatively planned drilling direction will be automatically reproduced, eliminating the needs for computerized and/or imaging systems during the surgical intervention.

As shown in FIG. 3, the first pair of setscrews 44 are prevented from being axially removed from the reference bone-engaging element 40 by means of a pair of threaded caps 60 screwed into a lateral mounting plate 62 secured to one side of the reference bone-engaging element 40 by means of a threaded fastener 64. A slot 66 is defined in each cap 60 for receiving a driving tool (not shown) to transmit a torque directly to the associated setscrew 44.

The intermediate support 42 includes a main body portion 68 and a top mounting plate 70. Conical threaded caps 72 are provided for securing the top mounting plate 70 to the main body portion 68 as well as for preventing axial withdrawal of the second pair of setscrews 46 from the intermediate support 42.

As shown in FIG. 4, two transferring cylinders 74a and 74b mounted between the top mounting plate 70 and the main body portion 68 of the intermediate support 42 are threadably engaged on respective setscrews 44 for axial movement along the threaded shank portion thereof. The cylinders 74a and 74b define respective diametrical threaded through bores (not shown) for receiving the corresponding setscrews 44 and are provided at opposed ends thereof with respective pivot pins 76a and 76b extending at right angles to the through bores for allowing the intermediate support 42 to pivot in the plane of the first pair of screws 44 in response to an axial displacement of one of the cylinders 74a/74b on the associated setscrew 44. The pivot pins 76a of the cylinder 74a are received in corresponding holes 78 defined in the top mounting plate 70 and the underlying surface of the main body portion 68 so as to form a single pivot between the cylinder 74a and the unified main body portion 68 and top mounting plate 70 of the intermediate support 42. Unlike the pivot pins 76a of the cylinder 74a, the pivot pins 76b of the cylinder 74b are received in respective slots 80 defined in the top mounting plate 70 and the underlying surface of the main body portion 68, thereby providing two degrees of movement between the cylinder 74b and the intermediate support 42. Indeed, the cylinder 74b will be allowed to pivot and slide relative to the intermediate support 42 in a plane parallel to the plane of the first pair of setscrews 44.

As shown in FIG. 3, slots 82 are defined in the conical caps 72 to allow the driving tool to engage the second pair of setscrews 46 and drive the same according to the adjustment parameters calculated by the software. The screws 46 extend through respective cylinders 84a and 84b mounted between a lateral mounting plate 86 and one side of the guide 36. The cylinders 84a and 84b are similar to cylinders 74a and 74b and include respective diametrical threaded through bores (not shown) for receiving the shank portion of the associated setscrews 46 and pivot pins 88a and 88b extending from respective opposed ends of the cylinders 84a and 84b along a pivot axis perpendicular to the axis of the through bores. The pivot pins 88a of the cylinder 84a are received in corresponding holes 90 defined in the lateral mounting plate 86 and the mounting plate facing side of the guide 36 so as to allow pivotal movement between the cylinder 84a and the guide 36 about the pivot axis defined by the pivot pins 88a, as shown in FIG. 4. The pivot pins 88b of the cylinder 84b are received in respective slots 92 defined in the guide and the lateral mounting plate 86 to allow pivotal and sliding movements between the cylinder 84b and the guide 36 in a plane parallel to the plane of the second pair of setscrews 46.

The above described adjusting mechanism formed by the first and second pairs of setscrews 44 and 46 and the associated cylinders 74 and 84 allow to adjust the orientation of the guide 36 so that the drilling axis defined thereby and the preoperatively calculated drilling direction match each other perfectly. The adjustment is effected by rotating the setscrews 44 and 46 in a given number of turns, which can be computed by the software as explained hereinbefore, different for each screw, to obtain a combination between a translation and a rotation.

The first adjustable bone-engaging element 48 is provided in the form a cylindrical finger 94 having a rounded distal end 98 for contacting a predetermined point on the inferior articular process 18 of the vertebra V. The finger 94 extends at right angles from a downwardly depending portion 100 of the main body portion 68 of the intermediate support 42. The length of the finger 94 is adjusted by operation of the setscrew 52 which is threadably received in the proximal end (not shown) of the finger 94. A cap 102 (FIG. 4) is provided for axially retaining the setscrew 52 in position in the guide.

The second adjustable bone-engaging element 50 includes an elongated stem portion 104 having a pair of bone-engaging fingers 106 extending in a V-shaped configuration from a distal end thereof. A planar web surface 108 is formed between the fingers 106 to provide a stable bearing point on the posterior surface of a corresponding transverse process 16 of the vertebra V. The opening angle of the fingers 106 is set so that the fingers 106 will respectively extend above and below the transverse process 16. The setscrew 54 (FIG. 4) is operable to adjust the length of the second adjustable bone-engaging element 50. A retaining cap 110 (FIG. 4) is threadably engaged with the guide 36 to axially retain the setscrew 54 in position therein.

As shown in FIG. 3, the reference bone-engaging element 40 is provided with a tail 112 adapted to be releasably secured to a clamping leg L1 of a surgical clamping tool T (FIG. 5) by means of threaded fasteners (not shown). A clamp adapter 114 is adapted to be releasably mounted to the other clamping leg L2 of the surgical clamping tool T to cooperate with the reference bone-engaging element 40 to maintain the surgical template 30 in position on the vertebra V after the template 30 has been properly located thereon with the bone-engaging elements 40, 48 and 50 matching the predetermined reference points on the vertebra V. The bone-engaging surfaces 58 and the adapter 114 will respectively be urged against the top and the undersurface of the spinal process 24 by the clamping mechanism of the surgical clamping tool T. The adapter 114 is provided with a curved bone-engaging surface 116 which is adapted to the general curvature of the undersurface of the spinal process 24.

In use, the setscrews are operated according to the adjustment parameters calculated by the software on the basis of the specific geometry of the vertebra in which a pilot hole has to be drilled. Once the bone-engaging element have been correctly configured and the guide properly oriented, the surgical tool is located on the vertebra in a unique predetermined position so that the bone-engaging element and the predetermined reference points on the vertebra are perfectly matched together, thereby automatically orienting the guide relative to the bone in accordance with the preoperative surgical planning. Then, the surgical template is releasably secured in position on vertebra using the surgical clamping tool T. Thereafter, the surgeon can drill the pilot hole by inserting a drilling bit through the passage 38 of the guide. After the drilling operation has been performed, the surgical template can be removed and readjusted in accordance to another modeled vertebra of a same patient or of another patient.

Although the present invention is primarily designed for assisting a surgeon in drilling a hole in a vertebra, it is understood that it could serve other purposes. For instance, the present invention could also be used for drilling, cutting and shaping various bones. Indeed, the guide does not necessarily have to be a drill guide but could consist of other types of guides depending on the medical task to be performed.

It is also understood that the guide 36 can be laterally mounted on the left side of the bone reference engaging element 42 with the associated linking elements for placement on the left side of the vertebra V.

1. A surgical template adjustable in conformity to specific geometric parameters of an intraoperatively reachable bone surface of a patient's bone, comprising positioning means including a number of bone-engaging elements adapted to be preoperatively adjusted and maintained in a predetermined configuration to match corresponding predetermined contact points on said bone surface for allowing said surgical template to be readily intraoperatively registered in a predetermined position on the bone surface, and a guide forming part of said surgical template for guiding a surgical tool in a predetermined direction to a specific location on the bone surface when the surgical template is in said predetermined position.

2. A surgical template as defined in claim 1, wherein said bone-engaging elements include a reference bone-engaging element, said guide being adjustably mounted to said reference bone-engaging element for movement between an unset position and a set position wherein said guide is aligned with said predetermined direction to guide the surgical tool to said specific location when said surgical template is in said predetermined position on the bone surface.

3. A surgical template as defined in claim 2, further including an adjuster operable for preoperatively adjusting the position and orientation of said bone-engaging elements and said guide according to the specific geometric parameters of the bone surface.

4. A surgical template as defined in claim 3, wherein said adjuster includes first and second pairs of parallel setscrews, and wherein said guide is connected to said reference bone-engaging element via an intermediate support, said first pair of parallel setscrews extending in a first plane and being mounted to said reference bone-engaging element to cooperate in translating and rotating said intermediate support relative to said reference bone-engaging element in a plane parallel to said first plane, said second pair of parallel setscrews extending in a second plane perpendicular to said first plane and being mounted to said intermediate support for translating and rotating said guide relative to said intermediate support in a plane parallel to said second plane.

5. A surgical template as defined in claim 4, wherein said bone-engaging elements further include a first adjustable bone-engaging element connected to said intermediate support for movement therewith.

6. A surgical template as defined in claim 5, wherein said adjuster further includes a first additional setscrew mounted to said intermediate support for linearly displacing said first adjustable bone-engaging element and adjusting the position thereof relative to said intermediate support.

7. A surgical template as defined in claim 4 or 5, wherein said bone-engaging element further includes a second adjustable bone-engaging element connected to said guide for movement therewith.

8. A surgical template as defined in claim 7, wherein said adjuster further includes a second additional setscrew mounted to said guide for linearly displacing said second adjustable bone-engaging element and adjusting the position thereof relative to said guide.

9. A surgical template as defined in claim 4, wherein first and second transferring members are threadably engaged on respective setscrews of said first set of parallel setscrews for movement therealong, and wherein said first transferring member is pivotally mounted to said intermediate support for pivotal movement about a fixed pivot axis normal to said first plane, whereas said second transferring member has a pivot normal to said first plane and engaged in a slot defined in said intermediate support.

10. A surgical template as defined in claim 9, wherein said slot extends perpendicularly to said first pair of setscrews in said first plane thereof.

11. A surgical template as defined in claim 4 or 9, wherein third and fourth transferring members are threadably engaged on respective setscrews of said second pair of parallel setscrews for movement therealong, and wherein said third transferring member is pivotally related to said guide for allowing pivotal movement therebetween about a fixed pivot axis normal to said second plane, whereas said fourth transferring member has a pivot normal to said second plane and engaged in a slot defined in said guide.

12. A surgical template as defined in claim 4, wherein said first plane is transversal to said reference bone-engaging member, and wherein said second plane extends laterally with respect to said reference bone-engaging element.

13. A surgical template as defined in claim 4, wherein said reference bone-engaging element includes first and second perpendicular pairs of bone-engaging surfaces, wherein the bone-engaging surfaces of each said first and second pairs extend in a V-shaped configuration.

14. A surgical template as defined in claim 2, further including securing means for releasably holding said surgical template in said predetermined position after said bone-engaging elements have been properly placed in contact with said corresponding predetermined contact points on said bone surface.

15. A surgical template as defined in claim 13, wherein said reference bone-engaging element is adapted to be mounted to a surgical clamping tool for releasably holding said surgical template in said predetermined position on the bone surface.

16. A surgical template as defined in claim 15, further comprising a bone-engaging adapter adapted to be mounted to a first clamping leg of the surgical clamping tool, and wherein said reference bone-engaging element is adapted to be mounted to a second clamping leg of the surgical clamping tool, said first pair of bone-engaging surfaces cooperating with said bone-engaging adapter to clamp the patient's bone.

17. The use of a surgical template as defined in any of claims 1 to 14 for orienting a drill in a preoperatively defined direction relative to the bone surface.

18. A method of orienting a surgical tool relative to a bone surface, wherein the surgical tool must contact a specific location on the bone surface at a predetermined angle, comprising the steps of: generating a three dimensional computer model of the bone surface, providing a surgical template having bone-engaging elements and a guide; given the specific geometrical parameters of the bone surface, adjusting said bone-engaging elements in a predetermined configuration in which said bone-engaging elements match predetermined contact points on said bone surface for allowing said surgical template to be registered in a unique preoperatively determined position on said bone surface; given said preoperatively determined position, adjusting the orientation of said guide according to the specific geometrical parameters of the bone surface and the task to be performed; localizing said templates on said bone surface until a perfect match is obtained with said bone-engaging elements abutting against said corresponding predetermined contact points on the bone surface, thereby automatically orienting said guide relative to said bone surface for guiding said surgical tool to contact the specific location on the bone surface at the predetermined angle.

19. A method as defined in claim 18, further comprising the steps of generating a computer model of the surgical template on the basis of the geometry of the bone surface.

20. A method as defined in claim 18, further comprising the step of: releasably securing said surgical template on the bone surface after the surgical template has been localized thereon.

21. A method as defined in claim 18, wherein said surgical template includes a number of setscrews which are operable for adjusting the bone-engaging elements and the guide, and wherein the steps of adjusting the bone-engaging elements and the guide are effected by first calculating, on the basis of the three dimensional computer model of the bone surface, the rotation that must be imparted to each setscrew, and then operating each setscrew accordingly.
